(19) United States
(12) Patent Application Publication
(10) Pub. No.: US 2018/0303746 A1
(43) Pub. Date: Oct. 25, 2018

(54) TREATMENT FOR SCALP DRYNESS AND CURLY HAIR

(71) Applicant: LivSo, LLC, Columbus, OH (US)

(72) Inventor: Shari HICKS-GRAHAM, Columbus, OH (US)

(73) Assignee: LivSo, LLC, Columbus, OH (US)

(21) Appl. No.: 15/883,369

(22) Filed: Jan. 30, 2018

Related U.S. Application Data
(60) Provisional application No. 62/487,873, filed on Apr. 20, 2017.

(51) Int. Cl. A61K 8/92; A61K 8/898; A61K 8/44; A61K 8/42; A61K 8/34; A61Q 5/00; A61Q 5/12

(52) U.S. Cl. CPC A61K 8/922; A61K 8/898; A61K 8/44; A61Q 5/12; A61K 8/342; A61Q 5/006; A61K 8/42

(57) ABSTRACT

A treatment for scalp dryness and curly hair including a shampoo composition, a conditioning composition and a moisturizing composition. The shampoo composition including about 5-50% by weight of a surfactant, at least 2% amino acids, about 5-15% by weight of an alcohol, and the remainder water. The conditioning composition including about 3%-15% by weight of a fatty alcohol, about 0.1-15% by weight of a conditioning agent, and the remainder water. The moisturizing lotion composition including about 0.1-12% by weight of an alcohol, about 0.1-10% by weight of a conditioning agent, and the remainder water.

This application claims priority to US Provisional Patent Application No. 62/487,873, filed on Apr. 20, 2017, which is incorporated herein in its entirety.

### BACKGROUND AND SUMMARY OF INVENTION

Dry scalp is a challenge faced by many people with curly or kinky hair. Naturally curly or kinky hair is prone to dryness for a variety of reasons. One of said reasons is the shape of the hair follicle. Tighter curls are very fragile and do not retain moisture as well as looser curls. The twists and turns of the curls prevent natural oils produced by the scalp from traveling all the way down the hair shaft resulting in dry hair. Another reason why curly or kinky hair is prone to dryness is the shape of the hair cuticles. The shape of curly or kinky hair tends to be more irregular. The ruffled cuticle edges make natural oil more difficult to track down the hair from the sebaceous glands of the scalp. The ruffled cuticles contribute to fragility and cause curly or kinky hair to often appear less shiny and dry. However, neither the shape of the hair follicle nor the structure of hair cuticles may be easily treated in order to relieve scalp dryness.

Several treatments claim to improve dry scalp dryness. Yet, many of these treatments involve chemical treatments that end up damaging the hair follicles often leading to hair breakage and even hair loss. Other remedies involve use of thick pomades. These pomades are used to cosmetically enhance the shine of curly hair. Unfortunately, thick pomades can often trap yeast and oil onto the scalp paradoxically leading to more dryness, scaling and irritation, which may exacerbate seborrhea or dandruff. As such, there is still a need for an effective way of treating scalp dryness in people with curly or kinky hair.

Currently disclosed is a method for treating scalp dryness and curly hair comprising the steps of: a) contacting the scalp and curly hair with a shampoo composition, said shampoo composition comprising: from about 5% to about 50% by weight of a surfactant, wherein said surfactant is an anionic surfactant, a sulfate-free surfactant or mixtures thereof, at least 2% amino acids; from about 5% to about 15% by weight of an alcohol, and the remainder water; b) rinsing the shampoo composition off from the scalp and curly hair; c) contacting the scalp and curly hair with a conditioning composition, said conditioning composition comprising: from about 3% to about 15% by weight of a fatty alcohol, from about 0.1% to about 15% by weight of a conditioning agent, and the remainder water; d) rinsing the conditioning composition off from the scalp and curly hair; e) contacting the scalp and curly hair with a moisturizing lotion composition, said moisturizing lotion composition comprising: from about 0.1 to 12% by weight of an alcohol, from about 0.1 to 10% by weight of a conditioning agent, and the remainder water; and f) leaving the moisturizing lotion composition on the scalp and curly hair.

The surfactant in the shampoo composition in the method for treating scalp dryness and curly hair may be coconut derived. The amino acid in the shampoo composition may be glutamic acid, aspartic acid, arginine, lysine or mixtures thereof.

The fatty alcohol in the conditioning composition may be cetyl alcohol, ceteratyl alcohol, panthenol, stearyl alcohol or mixtures thereof. The conditioning agent of the conditioning composition in the method for treating scalp dryness and curly hair may be amodimethicone, myristamidopropyl pg-dimonium chloride, natural based oil or mixtures thereof. The natural-based oil may further comprise Abyssinian seed oil, coconut oil, avocado oil, olive fruit oil, or mixtures thereof.

The conditioning agent of the moisturizing lotion composition in the method for treating scalp dryness and curly hair may be caprylic/capric triglyceride, willow bark extract, myristamidopropyl pg-dimonium chloride, ceramide, coconut oil or mixtures thereof.

Also disclosed is a treatment for treating scalp and curly hair comprising:

a shampoo composition comprising:
from about 5% to about 50% by weight of a surfactant, wherein said surfactant is an anionic surfactant, a sulfate-free surfactant or mixtures thereof,
at least 2% amino acids,
from about 5% to about 15% by weight of an alcohol, and
the remainder water;
   a conditioning composition comprising:
from about 3% to about 15% by weight of a fatty alcohol,
from about 0.1% to about 15% by weight of a conditioning agent, and
the remainder water; and
   a moisturizing lotion composition comprising:
from about 0.1 to 12% by weight of an alcohol,
from about 0.1 to 10% by weight of a conditioning agent, and
the remainder water.

The surfactant in the shampoo composition in the treatment for treating scalp dryness and curly hair may be coconut derived. The amino acid in the shampoo composition may be glutamic acid, aspartic acid, arginine, lysine or mixtures thereof.

The fatty alcohol in the conditioning composition may be cetyl alcohol, ceteratyl alcohol, panthenol, stearyl alcohol or mixtures thereof. The conditioning agent of the conditioning composition may be amodimethicone, myristamidopropyl pg-dimonium chloride, natural based oil or mixtures thereof. The natural-based oil may further comprise Abyssinian seed oil, coconut oil, avocado oil, olive fruit oil, or mixtures thereof.

The conditioning agent of the moisturizing lotion composition may be caprylic/capric triglyceride, willow bark extract, myristamidopropyl pg-dimonium chloride, ceramide, coconut oil or mixtures thereof.

### BRIEF DESCRIPTION OF THE DRAWINGS

The following is a brief description of the drawings accompanying this application to assist in further description of the embodiments of the invention:

FIG. 1 is a stability report for shampoo composition;

FIG. 2 is a stability report for conditioning composition; and

FIG. 3 is a stability report for moisturizing lotion composition.

### DETAILED DESCRIPTION OF THE DRAWINGS

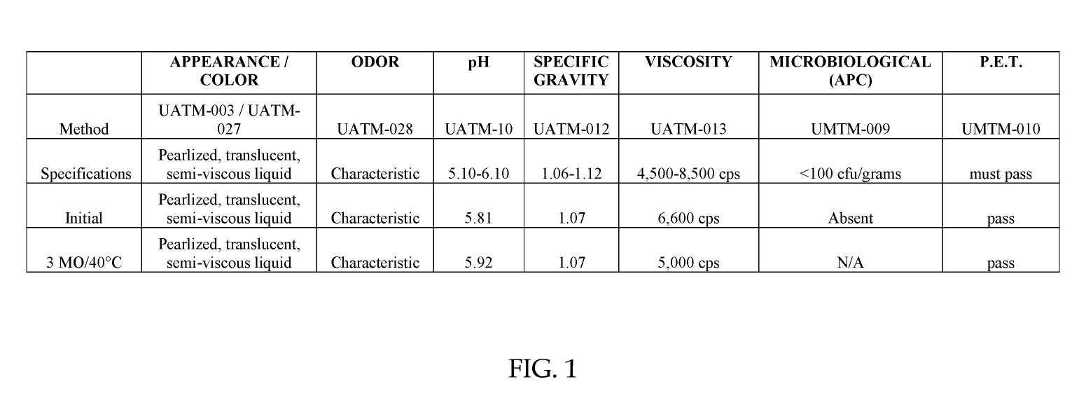

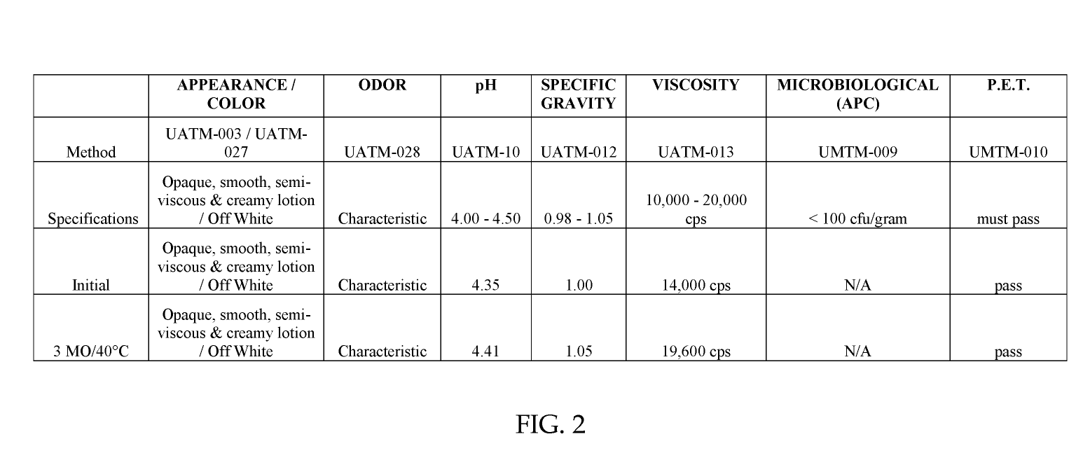

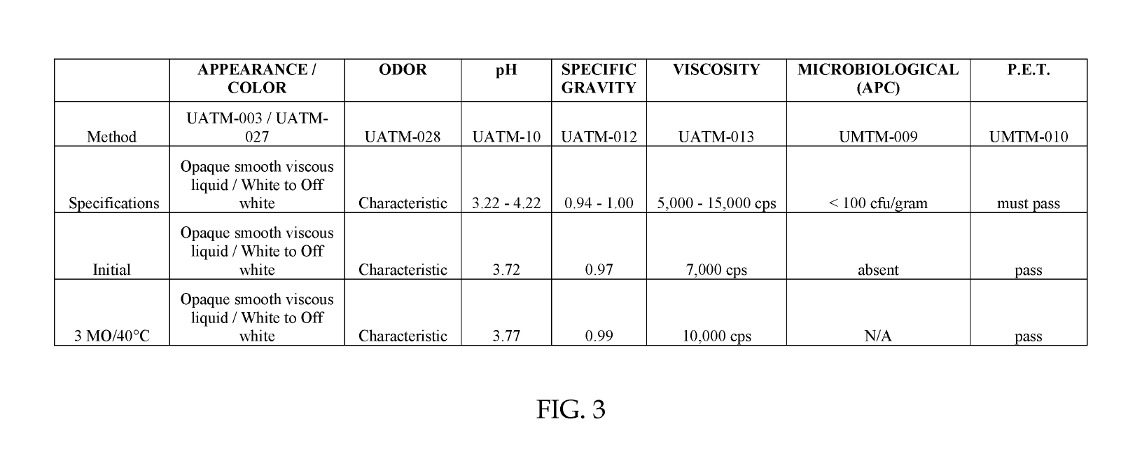

Referring to FIG. 1 to FIG. 3, stability protocols for a shampoo composition, for a conditioning composition and for a moisturizing lotion composition are illustrated. The appearance/color, odor, pH, specific gravity, viscosity, microbiological (APC) and P.E.T. were measured for each composition throughout a period of three months. As illustrated in FIG. 1, the shampoo composition had an appearance of a pearlized, translucent, semi-viscous liquid with an average pH of 5.87. As shown in FIG. 2, the conditioning composition had an appearance of an off-white opaque, smooth, semi-viscous and creamy lotion with an average pH of 4.38. As evident in FIG. 3, the moisturizing lotion composition had an appearance of a white to off-white opaque smooth viscous liquid with an average pH of 3.75. All three compositions passed the stability tests at the end of the three-month period.

The shampoo composition may comprise from about 5% to about 50% by weight of a surfactant, wherein said surfactant is an anionic surfactant, a sulfate-free surfactant or mixtures thereof, at least 2% amino acids; from about 5% to about 15% by weight of an alcohol, and the remainder water. The alcohol in the shampoo composition may be xylitol. Xylitol fights potential flakes and dryness. The shampoo composition may comprise glycolic acid. The glycolic acid gently exfoliates scalp and gives hair strength and softness at the same time. The glycolic acid also makes the hair more manageable and restores its luster. The surfactant in the shampoo composition may be coconut derived. Coconut revitalizes hair by adding all natural protective moisture.

The conditioning composition may comprise from about 3% to about 15% by weight of a fatty alcohol, from about 0.1% to about 15% by weight of a conditioning agent, and the remainder water. The conditioning agent of the conditioning composition may be amodimethicone, myristamidopropyl pg-dimonium chloride, natural based oil or mixtures thereof. The natural based oil may be Abyssinian oil. The Abyssinian oil provides hair with natural shine & moisture while detangling and adding necessary lipids. The Abyssinian oil also helps to restore the scalp for hair growth. The conditioning composition may also contain hydrolyzed oat protein, which surrounds each hair with a layer of protection guarding against heat and styling damage. The conditioning composition may also contain glycolic acid, which gives hair strength and softness at the same time.

The moisturizing lotion composition may comprise from about 0.1 to 12% by weight of an alcohol, from about 0.1 to 10% by weight of a conditioning agent, and the remainder water. The conditioning agent in the moisturizing lotion composition may be caprylic/capric triglyceride, willow bark extract, myristamidopropyl pg-dimonium chloride, ceramide, coconut oil or mixtures thereof. Ceramides moisturize and condition the scalp. Willow bark extract exfoliates the scalp to help remove flakes. Coconut oil restores scalp health and promotes hair growth.

While this invention has been shown and described with respect to a detailed embodiment thereof, it will be understood by those skilled in the art that changes in form and detail thereof may be made without departing from the scope of the claims of the invention.

What is claimed:

1. A method for treating scalp dryness and curly hair comprising the steps of:
a. contacting the scalp and curly hair with a shampoo composition, said shampoo composition comprising:
from about 5% to about 50% by weight of a surfactant, wherein said surfactant is an anionic surfactant, sulfate-free surfactant or mixtures thereof,
at least 2% amino acids;
from about 5% to about 15% by weight of an alcohol, and
the remainder water;
b. rinsing the shampoo composition off from the scalp and curly hair;
c. contacting the scalp and curly hair with a conditioning composition, said conditioning composition comprising:
from about 3% to about 15% by weight of a fatty alcohol,
from about 0.1% to about 15% by weight of a conditioning agent, and
the remainder water;
d. rinsing the conditioning composition off from the scalp and curly hair;
e. contacting the scalp and curly hair with a moisturizing lotion composition, said moisturizing lotion composition comprising:
from about 0.1 to 12% by weight of an alcohol,
from about 0.1 to 10% by weight of a conditioning agent, and
the remainder water; and
f. leaving the moisturizing lotion composition on the scalp and curly hair.

2. The method for treating scalp dryness and curly hair as claimed in claim 1 wherein the surfactant is coconut derived.

3. The method for treating scalp dryness and curly hair as claimed in claim 1 wherein the fatty alcohol in the conditioning composition is cetyl alcohol, ceteratyl alcohol, panthenol, stearyl alcohol or mixtures thereof.

4. The method for treating scalp dryness and curly hair as claimed in claim 1 wherein the conditioning agent of the conditioning composition is amodimethicone, myristamidopropyl pg-dimonium chloride, natural based oil or mixtures thereof.

5. The method for treating scalp dryness and curly hair as claimed in claim 4 wherein the natural-based oil is Abyssinian seed oil, coconut oil, avocado oil, olive fruit oil, or mixtures thereof.

6. The method for treating scalp dryness and curly hair as claimed in claim 1 wherein the conditioning agent in the moisturizing lotion composition is caprylic/capric triglyceride, willow bark extract, myristamidopropyl pg-dimonium chloride, ceramide, coconut oil or mixtures thereof.

7. The method for treating scalp dryness and curly hair as claimed in claim 1 wherein the amino acid is glutamic acid, aspartic acid, arginine, lysine or mixtures thereof.

8. A treatment for scalp dryness and curly hair comprising:
a shampoo composition comprising:
from about 5% to about 50% by weight of a surfactant is an anionic surfactant, a sulfate-free surfactant or mixtures thereof,
at least 2% amino acids,
from about 5% to about 15% by weight of an alcohol, and
the remainder water;
a conditioning composition comprising:
from about 3% to about 15% by weight of a fatty alcohol,
from about 0.1% to about 15% by weight of a conditioning agent, and
the remainder water; and
a moisturizing lotion composition comprising:
from about 0.1 to 12% by weight of an alcohol,
from about 0.1 to 10% by weight of a conditioning agent, and
the remainder water.

9. The treatment for scalp dryness and curly hair as claimed in claim 8 wherein the surfactant is coconut derived.

10. The treatment for scalp dryness and curly hair as claimed in claim 8 wherein the fatty alcohol is cetyl alcohol, ceteratyl alcohol, panthenol, stearyl alcohol or mixtures thereof.

11. The treatment for scalp dryness and curly hair as claimed in claim 10 wherein the conditioning agent of the conditioning composition is amodimethicone, myristamidopropyl pg-dimonium chloride, natural based oil or mixtures thereof.

12. The treatment for scalp dryness and curly hair as claimed in claim 8 wherein the natural-based oil is Abyssinian seed oil, coconut oil, avocado oil, olive fruit oil, or mixtures thereof.

13. The treatment for scalp dryness and curly hair as claimed in claim 8 wherein the conditioning agent in the moisturizing lotion composition is caprylic/capric triglyceride, willow bark extract, myristamidopropyl pg-dimonium chloride, ceramide, coconut oil or mixtures thereof.

14. The treatment for scalp dryness and curly hair as claimed in claim 8 wherein the amino acid is glutamic acid, aspartic acid, arginine, lysine or mixtures thereof.
